(19) United States
(12) Patent Application Publication
(10) Pub. No.: US 2020/0154801 A1
(43) Pub. Date: May 21, 2020

(54) Gown with Telemetry Pocket and One or More Flaps

(71) Applicant: Medline Industries, Inc, Northfield, IL (US)

(72) Inventors: Teri Burke, Wauconda, IL (US); Rachel Sinde, Des Plaines, IL (US); Sue Macinnes, Trout Valley, IL (US); Oscar Chavarria, Frisco, TX (US); Emily Somers, Trout Valley, IL (US)

(21) Appl. No.: 16/685,615

(22) Filed: Nov. 15, 2019

Related U.S. Application Data
(60) Provisional application No. 62/767,814, filed on Nov. 15, 2018.

(51) Int. Cl. A41D 13/12; A61B 46/20

(52) U.S. Cl. CPC A41D 13/1245; A61B 46/20

(57) ABSTRACT

A gown (100) includes a body-covering portion (102) having an interior side (402) and an exterior side (101). The body-covering portion also defines an aperture (301). A flap (118) is coupled to the exterior side. The flap includes one or more sides that are selectively attachable to the exterior side by one or more fasteners, as well as a double-paneled telemetry pocket (126). The flap covers the aperture when the one or more fasteners are fastened, and exposes the aperture when the one or more fasteners are unfastened and the flap is folded away from the exterior side.

### CROSS REFERENCE TO PRIOR APPLICATIONS

This application claims priority and benefit under 35 U.S.C. § 119(e) from U.S. Provisional Application No. 62/767,814, filed Nov. 15, 2018, which is incorporated by reference for all purposes.

### BACKGROUND

### Technical Field

This disclosure relates generally to gowns, and more particularly to gowns for medical procedures.

### Background Art

Patients frequently wear gowns during medical procedures. The gowns allow medical professionals to access parts of the body under treatment, while at the same time preserving patient dignity by keeping the patient covered. Access can be difficult, however, when the patient is coupled to medical equipment, some of which may not be detachable from the patient. It would be advantageous to have an improved gown that allows easier access to a patient coupled to medical equipment.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates a front elevation view of one explanatory gown, with an exterior side of a body-covering portion exposed, in accordance with one or more embodiments of the disclosure.

FIG. 2 illustrates a rear elevation view of the gown of FIG. 1.

FIG. 3 illustrates a front elevation view of the gown of FIG. 1 with a flap covering an aperture and including thereon a double-paneled telemetry pocket folded down.

FIG. 4 illustrates a rear elevation view of the gown of FIG. 1 with the left and right flaps opened such that the interior side of the body-covering portion can be seen.

FIG. 5 illustrates a front elevation view of the gown of FIG. 1, with an interior side of the body-covering portion exposed, in accordance with one or more embodiments of the disclosure.

FIG. 6 illustrates a rear elevation view of the gown of FIG. 5.

FIG. 7 illustrates a rear elevation view of the gown of FIG. 5 with the left and right flaps opened such that the interior side of the body-covering portion can be seen.

FIG. 8 illustrates one explanatory double-paneled telemetry pocket in accordance with one or more embodiments of the disclosure.

FIG. 9 illustrates one explanatory gown sleeve in accordance with one or more embodiments of the disclosure.

FIG. 10 illustrates another explanatory gown sleeve in accordance with one or more embodiments of the disclosure.

FIG. 11 illustrates one explanatory method of manufacturing a gown in accordance with one or more embodiments of the disclosure.

FIG. 12 illustrates various embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions.

As used herein, the terms “substantially”, “essentially”, “approximately”, “about” or any other version thereof, are defined as being close to as understood by one of ordinary skill in the art, and in one non-limiting embodiment the term is defined to be within ten percent, in another embodiment within five percent, in another embodiment within one percent and in another embodiment within one half of one percent. The term “coupled” as used herein is defined as connected, although not necessarily directly and not necessarily mechanically. Also, reference designators shown herein in parenthesis indicate components shown in a figure other than the one in discussion. For example, talking about a device (10) while discussing figure A would refer to an element, 10, shown in figure other than figure A.

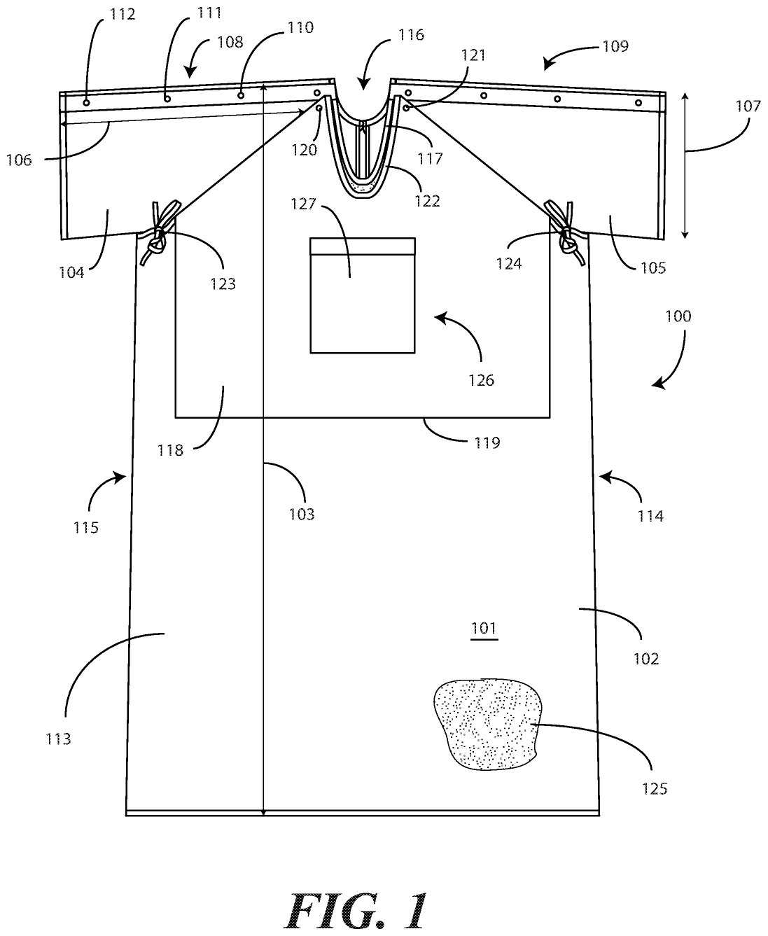

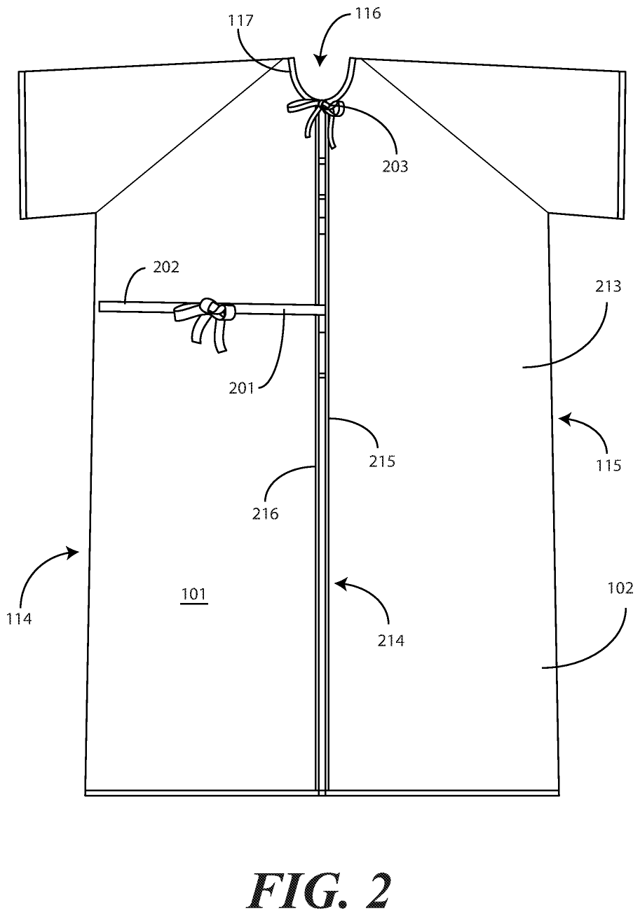

Turning now to FIGS. 1 and 2, illustrated therein is one explanatory embodiment of a gown 100 configured in accordance with one or more embodiments of the disclosure. In FIG. 1, the gown 100 is folded such that an exterior side 101 of the gown 100 is exposed outwardly. The exterior side 101 can be ornamented with a first pattern or color 125.

In one or more embodiments, the gown 100 includes a body-covering portion 102 that is configured to wrap about the torso of a wearer. FIG. 1 illustrates a front elevation view of the gown 100 with its exterior side 101 of the body-covering portion 102 exposed, while FIG. 2 illustrates the rear elevation view thereof.

In one embodiment, the gown 100 is manufactured from a washable fabric, such as cotton or polyester. The body-covering portion 102 can also be manufactured from a durable, washable fabric such as a cotton-polyester blend. In another embodiment, the gown 100 could be manufactured from non-woven fabric so as to be disposable.

The body-covering portion 102 of the gown 100 can optionally include multiple layers, such as a textile layer manufactured from a cotton-polyester blend and a water resistant lining coupled thereto that prevents the passage of fluids through the body-covering portion 102. Similarly, antimicrobial or antibacterial coatings or treatments may be applied to the material forming the gown 100 as well. Other materials suitable for manufacturing the body-covering portion 102 will be obvious to those of ordinary skill in the art having the benefit of this disclosure.

The gown 100 can be configured in a variety of lengths. In the illustrative embodiment of FIGS. 1-2, the gown 100 is configured with a length 103 that runs from a wearer's shoulder to below their knee. In one or more embodiments, the length 103 is between forty-eight and fifty inches, with one example of length 103 being about and 48.5 inches. It will be clear to those having the benefit of this disclosure that such a length 103 is illustrative only, as the gown 100 can be configured with other lengths without departing from the spirit and scope of the disclosure. Moreover, it should be noted that the various sides of the gown 100 do not have to be the same length. For example, the front of the gown 100, shown in FIG. 1, can be longer or shorter than the back of the gown 100, shown in FIG. 2, or vice versa.

In one or more embodiments, a first sleeve 104 and a second sleeve 105 extend distally from the body-covering portion 102. In this illustrative embodiment, the first sleeve 104 and the second sleeve 105 are short sleeves. However, it will be clear to those of ordinary skill in the art having the benefit of disclosure that embodiments of the invention are not so limited. The gown 100 may equally be configured with long sleeves or no sleeves has a particular application may warrant. In one or more embodiments, the length 106 of the first sleeve 104 and the second sleeve 105 is about 17.25 inches. The sleeve opening 107 may measure, for example twenty-three inches or 24.5 inches.

The first sleeve 104 and the second sleeve 105 are configured to receive the arms of a wearer when the gown 100 is donned. In one or more embodiments, one or both of the first sleeve 104 or the second sleeve 105 is configured so that a medical services provider can open the sleeves to reveal the patient's arm, as necessary, for medical examination. In one or more embodiments, the first sleeve 104 and the second sleeve 105 are configured as what is known as “intravenous sleeves” or “IV sleeves,” in that they have openings 108,109 through which intravenous medical apparatuses may pass. The openings 108,109 may have their own closing mechanism. In one or more embodiments, the openings 108,109 extend down the side of one or both sleeves.

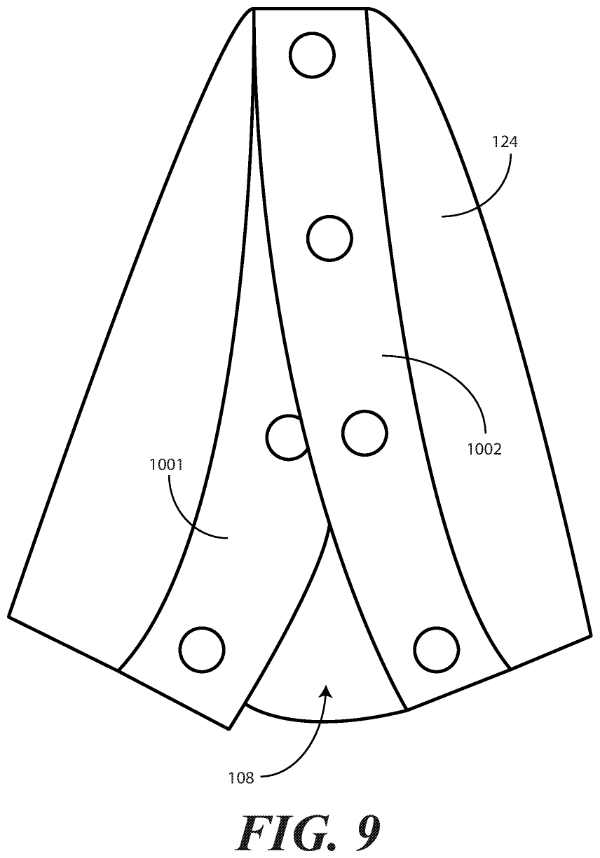

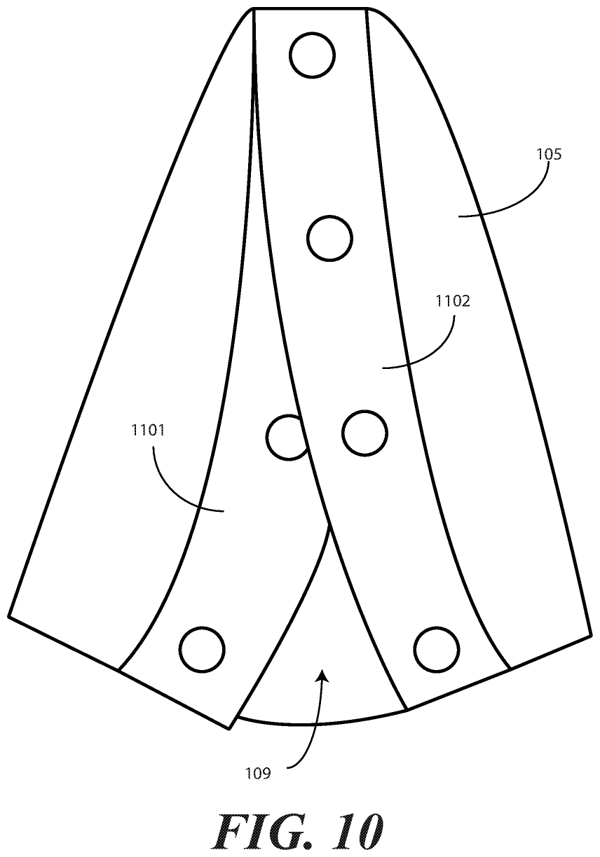

In one or more embodiments, the openings 108,109 can include a closing mechanism. The closing mechanisms may be coupled to a border that runs along each edge of the opening. For example, opening 108 can include a left border and a right border, while opening 109 also includes a left border and a right border as well. In one or more embodiments, the left border and the right border of the first sleeve 104 are colored with a first color, while the left border and the right border of the second sleeve 105 are colored with a second color that is different from the first color. This will be explained in more detail below with reference to FIGS. 9-10.

In the illustrative embodiment of FIGS. 1-2, the closing mechanisms comprise one or more snaps 110,111,112, which are disposed on either side of the openings 108,109. In other embodiments, the closing mechanisms comprise hook and loop fasteners, zippers, buttons, or other closing devices. The inclusion of the snaps 110,111,112 allows the same to be adequately spaced so as to advantageously allow intravenous tubing to pass between the snaps 110,111,112 and attached to a patient when wearing the gown 100. In other embodiments, the openings 108,109 can be omitted with the upper portion of the first sleeve 104 and the second sleeve 105 being closed by a perdurable connection, such as a seam.

In one embodiment, the body-covering portion 102 includes a front portion 113 and a rear portion 213. In one or more embodiments, the front portion 113 is configured for placement against the front of the torso of a patient when the patient is wearing the gown 100. The front portion 113 can be configured with varying widths, one example of which is about thirty inches. The body-covering portion 102 then wraps around the torso and terminates at an opening 214.

In one or more embodiments, the opening 214 is situated along the rear portion 213. In one or more embodiments, the opening 214 is closed when a first closure side edge 215 overlaps a second closure side edge 216. While the opening 214 is shown along the rear portion 213 in this illustrative embodiment, it should be noted that it could also be positioned along the front portion 113, a first side portion 114, or a second side portion 115 as well. In the illustrative embodiment of FIGS. 1-2, the opening 214 is configured to run the length 103 of the body-covering portion 102, up the rear portion 213 of the gown 100, and then terminating at a head insertion aperture 116, which serves as a neck opening through which a patient may place their neck when donning the gown 100.

To don the gown 100, a patient opens the opening 214 by pulling the first closure side edge 215 away from the second closure side edge 216. The patient then passes the first closure side edge 215 and the second closure side edge 216 about either side of their torso. The patient inserts their arms into the first sleeve 104 and the second sleeve 105, respectively. The patient then passes the second closure side edge 216 over the first closure side edge 215 so that a first section of the rear portion 213 of the body-covering portion 102 overlaps another section of the rear portion 213 of the body-covering portion 102.

The patient can then secure the gown 100 about the torso with one or more ties 201,202 and/or a neck tie 203. In this illustrative embodiment, a first tie 201 is coupled to, and extends distally from, the first closure side edge 215, while a second tie is coupled to, and extends distally from, the first side portion 114. The first tie 201 can be tied to the second tie 202 to close the opening 214 on the backside of the gown 100.

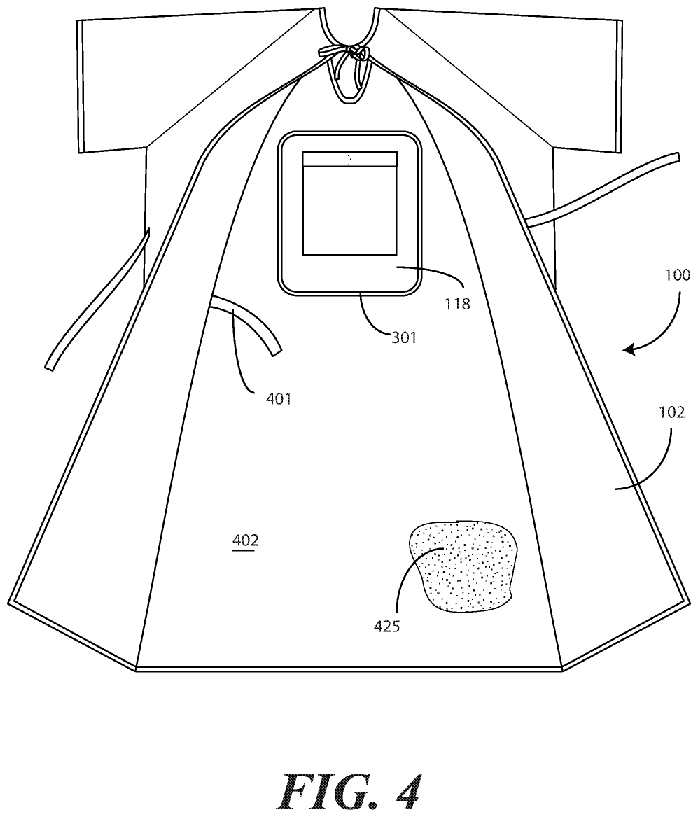

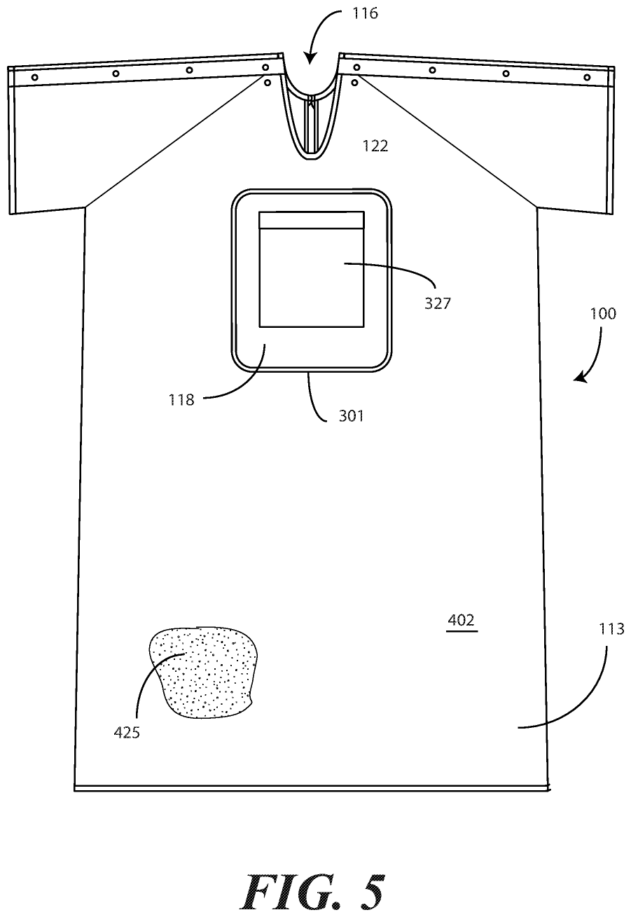

As shown in FIG. 4, an extra tie 401 may be included on the interior side 402 of the gown 100 to allow for the opening 214 to be closed when the gown 100 is turned inside out, as will be described below with reference to FIGS. 5-7. While one neck tie 203 is shown in FIGS. 1-2, in another embodiment multiple neck ties may be provided to allow the head insertion aperture 116 to be adjusted in size. The ties may be substituted with snaps, buttons, strips of hook-and-loop fasteners, or other materials.

Where the body-covering portion 102 is manufactured from a washable material, one or both of the ties 201,202 and/or neck tie 203 may also be manufactured from a durable, washable material. For example, in one embodiment one or both of the ties 201,202 and/or neck tie 203 are each manufactured from twill. One or both of the ties 201,202 and/or neck tie 203 can be manufactured from the same material as the body-covering portion 102, or from different material One or both of the ties 201,202 and/or neck tie 203 can be reinforced, such as with a layer of reinforcing material or with reinforcement stitching.

The ties 201,202 and/or neck tie 203 can be coupled or disposed on the body-covering portion 102 in a variety of ways. Where the body-covering portion 102 is manufactured from a washable material, the ties 201,202 and/or neck tie 203 can be sewn to the body-covering portion 102. Where the body-covering portion 102 is manufactured from a disposable material, the ties 201,202 and/or neck tie 203 can be attached by adhesives, thermal bonding, or other coupling processes.

In this illustrative embodiment, the head insertion aperture 116 defines a V-shaped opening. In one or more embodiments, the V-shaped opening may be lined or edged with a hem 117. While the head insertion aperture 116 could be round or take other shapes, the V-shape works to reduce pinching or gathering about the patient's neck.

In this illustrative embodiment, the exterior side 101 of the gown 100 comprises a flap 118. In one or more embodiments, the flap 118 is secured at its base to the body-covering portion 102 by a seam 119. In one or more embodiments, the top portion of the flap 118 is then selectively attachable to the exterior side 101 of the body-covering portion 102 by one or more fasteners 120,121. In this illustrative embodiment, the one or more fasteners 1201,121 comprise snaps that selectively attach the top portion of the flap 118 to the neckline 122 of the head insertion aperture 116 at the top of the openings 108,109 of the first sleeve 104 and the second sleeve 105, respectively. Advantageously, locating the snaps in this location prevents them from showing up on radiographs or other imagery captured of a patient's chest while the patient is wearing the gown 100. While two snaps are shown in FIG. 1, more or fewer snaps could be used in other embodiments. Additionally, in other embodiments the snaps can be replaced by other fasteners, including hook and loop fasteners, zippers, and so forth.

It should be noted that while one flap 118 is shown in FIG. 1 and in subsequent figures, embodiments of the disclosure are not so limited. In other embodiments, the gown 100 can include a second flap. In one or more embodiments, the second flap is coupled to the interior side of the gown 100. In one or more embodiments, the second flap comprises one or more second flap sides that are selectively attachable to the interior side by one or more other fasteners. In one or more embodiments, the second flap covers at least another telemetry pocket when the one or more other fasteners are fastened, and exposes the at least another telemetry pocket when the one or more other fasteners are unfastened and the second flap is folded away from the interior side, and so forth.

As illustrated in FIG. 1, side portions of the flap 118 are coupled to the exterior side 101 of the gown 100 by two side ties 123,124. In one or more embodiments, each side tie 123,124 includes a first tie member affixed to the armpit-covering region of the gown 100 and a second tie member affixed to the flap 118. The use of side ties 123,124 to attach the flap 118 to the exterior side 101 of the gown 100 advantageously prevents hard components, such as snaps, zippers, or buttons, from appearing on radiographs or other imagery captured of a patient's chest while the patient is wearing the gown 100. As with the snaps at the neckline 122, while two side ties 123,124 are shown in the illustrative embodiment of FIG. 1, more or fewer side ties could be included to couple the flap 118 to the exterior side 101 of the gown 100 at more or fewer locations, and so forth.

In one or more embodiments, the flap 118 is configured to substantially cover the chest of a patient who is wearing the gown 100. Illustrating by example, in one or more embodiments the seam 119 coupling the base of the flap 118 to the exterior side 101 of the gown 100 has a width of about twenty-four inches. In this illustrative embodiment, a first left side member of the flap 118 extending from the left end of the seam 119 to the left side tie 123 then has a length of about twelve inches. A second left side member extending from the side tie 123 to the left fastener 120 then measures about twelve inches as well. Similarly, a first right side member of the flap 118 extending from the right end of the seam 119 to right side tie 124 has a length of about twelve inches, while a second right side member extending from the side tie 124 to the right fastener 121 then measures about twelve inches. These dimensions are illustrative only, as numerous others will be obvious to those of ordinary skill in the art having the benefit of this disclosure.

In this illustrative embodiment, only the exterior side 101 of the gown 100 includes the flap 118. As will be described in more detail below with reference to FIGS. 5-7, when the gown 100 is folded such that the interior side (402) of the body-covering portion 102 is exposed, only an aperture in the front portion 113 of the body-covering portion 102 will be visible, with the inner surface of the flap 118 exposed through this aperture when the flap 118 is attached to the exterior side 101 of the body-covering portion 102 using the fasteners 120,121 and the side ties 123,124.

Having the flap 118 affixed only to the exterior side 101 of the gown 100 means that the fasteners 120,121 and side ties 123,124 are exposed to the environment and are accessible by the patient or a health care services provider only when the exterior side 101 of the gown 100 is also exposed to the environment. When the gown 100 is folded such that the interior side (402) of the body-covering portion 102 is exposed, i.e., when being worn “inside out,” in one or more embodiments the fasteners 120,121 and side ties 123,124 are generally not readily accessible due to the fact that they are positioned between the body-covering portion 102 and/or neckline 122 of the head insertion aperture 116 and the body of the patient.

In this illustrative embodiment, the exterior side 101 is referred to as the “high risk side” due to the fact that it includes the flap 118 with both the fasteners 120,121 and side ties 123,124 exposed. A patient who is at high risk of needing medical attention requiring access to the patient's chest would wear the gown 100 as shown in FIGS. 1-2 with the exterior side 101 exposed, thereby allowing medical personnel to quickly and easily access both the fasteners 120,121 and the side ties 123,124 to decouple the sides and upper portion of the flap 118 from the exterior side 101 of the gown 100 to fold it down for chest access.

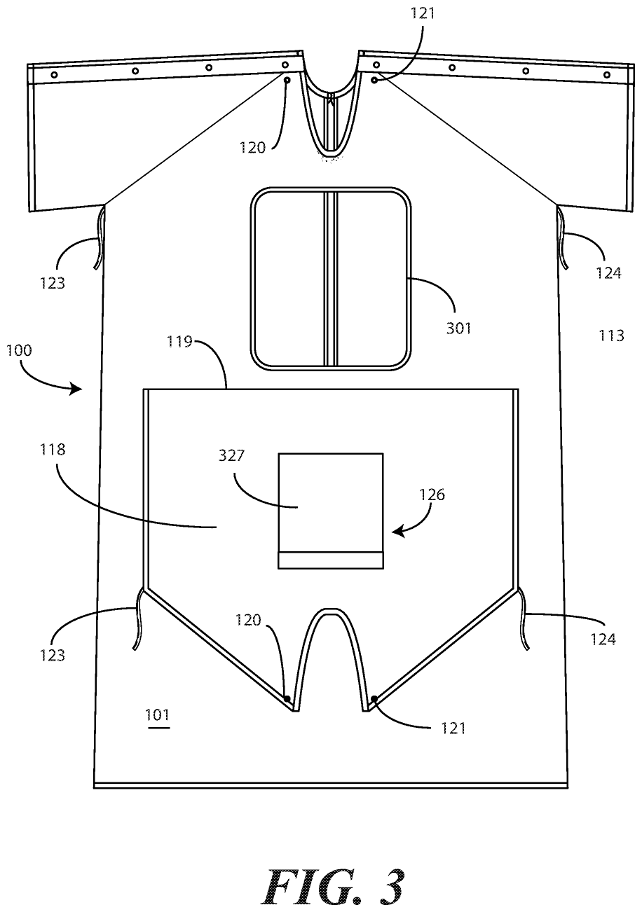

Turning briefly to FIG. 3, illustrated therein is the gown 100 in such a configuration. Specifically, the side ties 123,124 have been untied, and the fasteners 120,121 have been unfastened. This allows the flap 118 to be folded away from the upper portion of the front portion 113 of the exterior side 101 of the gown 100 at the seam 119, thereby revealing an aperture 301 that allows medical personnel to access the chest of a patient wearing the gown 100. Such medical personnel may be checking or adjusting medical equipment attached to the chest of the patient, performing cardiopulmonary resuscitation, or taking other action requiring access to the patient's chest. Once this procedure is complete, the side ties 123,124 can be retied untied, and the fasteners 120,121 refastened after the flap 118 is folded toward the upper portion of the front portion 113 of the exterior side 101 of the gown 100 at the seam 119. This both conceals the aperture 301, as was shown in FIG. 1, and also attaches the flap 118 to the upper portion of the front portion 113 of the exterior side 101 of the gown 100 as previously described.

As shown in FIG. 4, in one or more embodiments the interior side 402 of the gown 100 is referred to as the “low risk side” in that the interior side 402 does not include a separate flap. Instead, the aperture 301 defined by the body-covering portion 102 is continually visible from the interior side 402 of the gown 100, with the flap 118 exposed within the perimeter of the aperture 301 when the flap 118 is attached to the upper portion of the front portion (113) of the exterior side (101) of the gown 100 using the side ties (123,124) and the fasteners (120,121). When the side ties (123,124) are untied, and the fasteners (120,121) are unfastened, this allows the flap 118 to be folded away from the upper portion of the front portion (113) of the exterior side (101) of the gown 100 at the seam (119), which allows medical personnel to see through the aperture 301.

In one or more embodiments, the gown 100 can be color-coded to indicate which side is the high-risk side and which side is the low risk side. Illustrating by example, the hem at the neckline (122) of the head insertion aperture 116 could be color-coded to indicate which side is the high-risk side and which side is the low risk side. In another embodiment, the interior side 402 of the gown 100 can be a first color while the exterior side (101) of the gown 100 is a second color.

In another embodiment, the interior side 402 of the gown 100 can be configured in a first pattern, while the exterior side (101) of the gown 100 is a second pattern. Additionally, the flap 118 can be a different color or pattern (or combination of both) to indicate that the side with the flap 118 is the high-risk side while the side without the flap is the low risk side, and so forth.

In still other embodiments, printing can be included on the exterior side (101) or on the outside of the flap 118 to indicate that the exterior side (101) is the high-risk side. Similarly, in one or more embodiments printing can be included on the interior side 402 or on the inside of the flap 118 to indicate that the interior side 402 is the low-risk side. In other embodiments, the exterior side (101) can be configured with a first pattern or color (125), while the interior side 402 is configured with a second pattern or color 425 to distinguish the high-risk side from the low-risk side. Illustrating by example, in one embodiment the first pattern or color (125) may be a blue patterned material with crosses interspaced within a pattern of dark blue crossing lines while the second pattern or color 425 can be a purple patterned material with seashells or mountain peaks interspaced within another pattern of white crossing lines to distinguish the high-risk side from the low-risk side. Other examples of the first pattern or color (125) and the second pattern or color 425 will be obvious to those of ordinary skill in the art having the benefit of this disclosure.

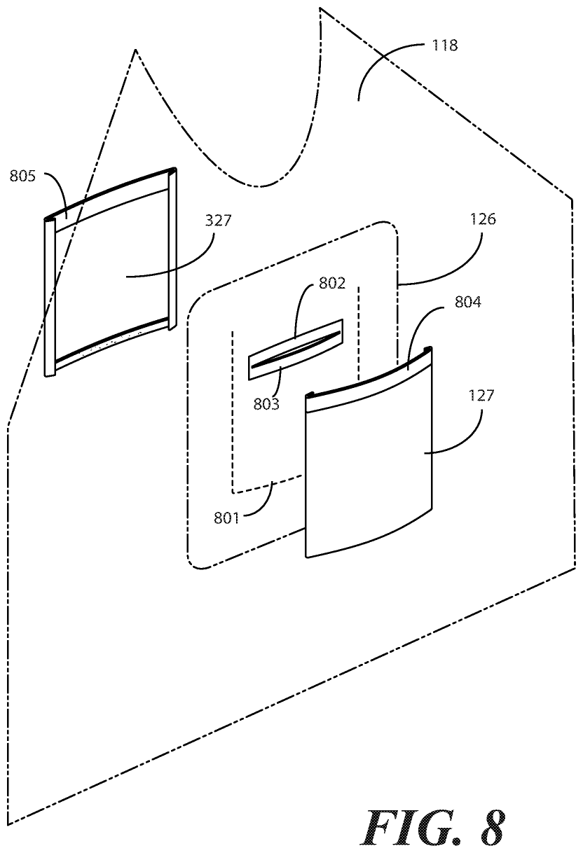

As shown in FIGS. 1 and 3, in one or more embodiments, the flap 118 of the gown 100 includes a double-paneled telemetry pocket 126. The double-paneled telemetry pocket 126 is referred to as being “double-paneled” because it includes a first pocket panel 127 on a first side of the flap 118, as shown in FIG. 1, and a second pocket panel 327 on a second side of the flap 118, as shown in FIG. 3. Turning now to FIG. 8, illustrated therein is an exploded view of this double-paneled telemetry pocket 126.

As shown in FIG. 8, the double-paneled telemetry pocket 126 includes a first pocket panel 127 coupled at a three-sided connection location 801 on a first side of the flap 118 and a second pocket panel 327 coupled at the three-sided connection location 801 on the second side of the flap 118. The flap 118 then defines an aperture 802 through which wires may be threaded when a transmitter or other medical device is positioned between the first pocket panel 127 and the first side of the flap 118, or alternatively between the second pocket panel 327 and the second side of the flap 118. While the aperture 802 shown as a horizontal slit with abutting sides in FIG. 8, the aperture 802 could be a vertical slit with abutting sides as well. In other embodiments, the aperture 802 will be configured as an open aperture with sides that do not abut. In still other embodiments, the aperture 802 can include overlapping flaps that close the aperture 802, but that can be separated to open the same.

In one or more embodiments, each of the first pocket panel 127 and the second pocket panel 327 is configured with a size sufficient to hold a telemetry monitor, other medical equipment, or personal items such as notes or papers. In one or more embodiments, each of the first pocket panel 127 and the second pocket panel 327 has a width of about six inches and a height of about seven and a half inches. In one or more embodiments, the aperture 802 has a length of about five inches. The aperture 802 can be reinforced with a border material 803 in one or more embodiments. Similarly, each of the first pocket panel 127 and the second pocket panel 327 can include a reinforcing border 804,805 coupled to the top, open side of each of the first pocket panel 127 and the second pocket panel 327.

In one or more embodiments, the first pocket panel 127 is exposed and accessible such that contents can be placed between the first side of the flap 118 and the first pocket panel 127 when the exterior side (101) of the gown (100) to which the flap 118 is attached is exposed to the environment and the flap 118 is attached to the exterior side (101) of the body-covering portion (102) using the fasteners (120,121) and the side ties (123,124). By contrast, the second pocket panel 327 will be exposed and accessible through the aperture (301) such that contents can be placed between the second side of the flap 118 and the second pocket panel 327 when the gown (100) is folded such that the interior side (402) of the body-covering portion (102) is exposed with the flap 118 is attached to the exterior side (101) of the body-covering portion (102) using the fasteners (120,121) and the side ties (123,124).

Thus, when a patient is wearing the gown (100) with the high-risk side facing outward, they can place contents between the first side of the flap 118 and the first pocket panel 127 by accessing the double-paneled telemetry pocket 126 from the first side of the flap 118. When wearing the gown (100) with the low-risk side facing outward, they can place contents between the second side of the flap 118 and the second pocket panel 327 by accessing the double-paneled telemetry pocket 126 from the second side of the flap 118 through the aperture (301) defined by the body-covering portion (102).

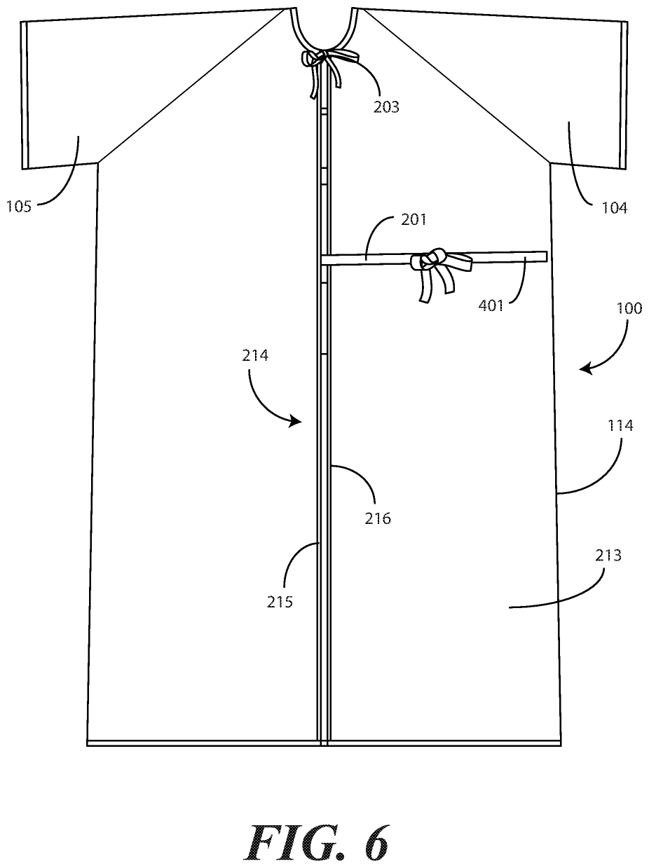

Turning now to FIGS. 5 and 6, illustrated therein is the gown 100 folded into a second configuration. Specifically, in FIGS. 5-6 the gown 100 is folded such that the interior side 402 of the gown 100 is exposed outwardly. Said differently, the gown 100 of FIGS. 5-6 is configured to be “inside out” compared to being configured “outside out” in FIGS. 1-2. As noted above, the interior side 402 can be ornamented with a second pattern or color 425.

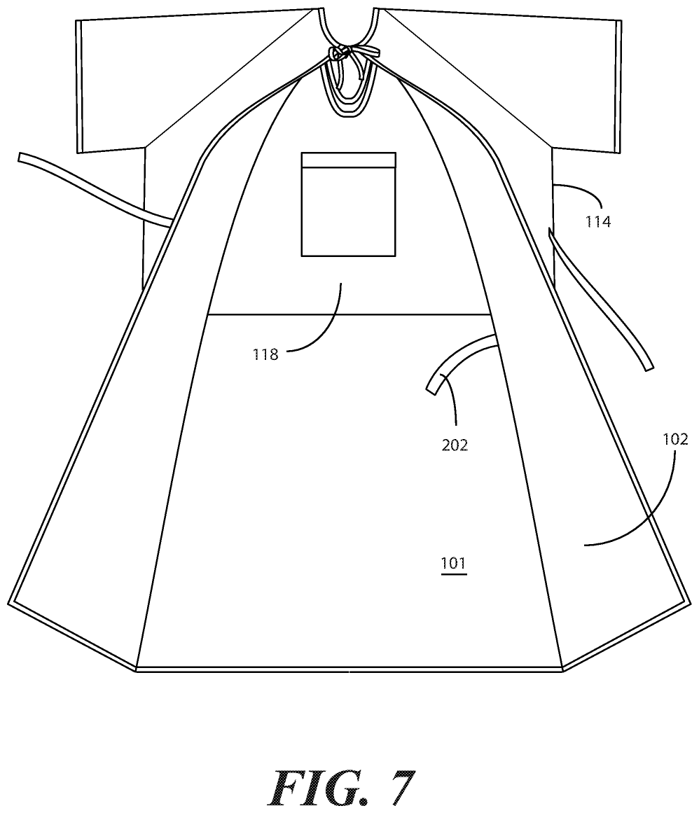

As before, the opening 214 is situated along the rear portion 213 of the gown 100. In one or more embodiments, the opening 214 is closed when a first closure side edge 215 overlaps a second closure side edge 216. Since the gown 100 is turned inside out, a patient can secure the gown 100 about the torso by tying the first tie 201, which is coupled to and extends distally from the first closure side edge 215, to the extra tie 401 that was shown as being attached to, and extending distally away from, the first side portion 114. As shown in FIG. 7, the original second tie 202, coupled to the exterior side 101 at the first side portion 114, is now situated between the body-covering portion 102 and the patient.

Recall from above that in one or more embodiments the exterior side 101 of the gown 100 comprises a flap 118, which is also shown in FIG. 7. Also recall from above that in one or more embodiments, only the exterior side 101 of the gown 100 includes the flap 118. Referring again to FIGS. 5-6, when the gown 100 is folded such that the interior side 402 of the body-covering portion 102 is exposed, this results in the aperture 301 defined by the front portion 113 of the body-covering portion 102 being visible. Within the perimeter of this aperture 301, both the inner surface of the flap 118 and the second pocket panel 327 are exposed when the flap 118 is attached to the exterior side (101) of the body-covering portion 102 using the fasteners (120,121) and the side ties (123,124).

Thus, when the gown 100 is being worn with the low-risk side outward, having the flap 118 affixed only to the exterior side (101) of the gown 100 means that the fasteners (120,121) and side ties (123,124) that couple the flap 118 to the exterior side (101) of the gown 100 are generally not readily or easily accessible due to the fact that they are positioned between the body-covering portion 102 and/or neckline 122 of the head insertion aperture 116 and the body of the patient. In emergency situations, medical personnel can still access these elements to detach the flap 118 from the body-covering portion (102), albeit with it requiring a bit of effort. For example, medical personnel can reach over the neckline 122 and/or through the aperture 301 to untie the side ties (123,124) and unfasten the fasteners (120,121) to pull the flap 118 through the aperture 301 to access the patient's chest. However, since there is a low risk that this need will arise due to the fact that the gown 100 is being worn with the low-risk side out, the need to perform this operation is unlikely.

Thus, to reiterate, in this illustrative embodiment the interior side 402 of the gown 100 is referred to as the “low risk side” in that the interior side 402 does not include a separate flap. Instead, the aperture 301 defined by the body-covering portion 102 is continually visible from the interior side 402 of the gown 100, with the flap 118 exposed within the perimeter of the aperture 301 when the flap 118 is attached to the upper portion of the front portion 113 of the exterior side (101) of the gown 100 using the side ties (123,124) and the fasteners (120,121).

Since the gown 100 can be worn with the high-risk side out, as shown above in FIGS. 1-2, and also with the low-risk side out, as shown in FIGS. 5-6, in one or more embodiments color coding can be used to assist users, for example, in determining how to put the gown together. For example, the side ties (123,124) can be a first color, with the first tie 201, the second tie (202), and the extra tie 401, as well as the neck ties 203 can be a second color such that one does not inadvertently tie ties of different colors together. Additionally, where the first sleeve 104 and the second sleeve 105 are configured as IV sleeves, they can be color-coded as well. Turning now to FIGS. 9-10, illustrated therein is one example of how this can be done.

The first sleeve 104 is shown in FIG. 9, while the second sleeve 105 is shown in FIG. 10. As noted above, the first sleeve 104 and the second sleeve 105 are configured to receive wearer's arms when the gown 100 is donned.

In one or more embodiments, one or both of the first sleeve 104 or the second sleeve 105 is configured so that a medical services provider can open the sleeves to reveal the patient's arm, as necessary, for medical examination. In one or more embodiments, the first sleeve 104 and the second sleeve 105 are configured as “IV sleeves” in that they have openings 108,109 through which intravenous medical apparatuses may pass. As previously described, the openings 108,109 may have their own closing mechanism. In one or more embodiments, the openings 108,109 extend down the side of one or both sleeves.

In FIGS. 9-10, each opening 108,109 includes a border to which the closing mechanisms are attached. For example, opening 108 includes a left border 901 and a right border 902, while opening 109 includes a left border 1001 and a right border 1002 as well. In one or more embodiments, the left border 901 and the right border 902 of the first sleeve 104 are colored with a first color, while the left border 1001 and the right border 1002 of the second sleeve 105 are colored with a second color that is different from the first color. Illustrating by example, in one or more embodiments the left border 901 and the right border 902 of the first sleeve 104 are colored orange, while the left border 1001 and the right border 1002 of the second sleeve 105 are colored white.

Where included, this color-coding advantageously functions to identify which closing element disposed along which border is normally coupled to a complementary closing element. Since closing elements attached to the left border 901 and the right border 902 of the first sleeve 104 are colored with a first color, one will not be inclined to attach them to closing elements attached to the left border 1001 and the right border 1002 of the second sleeve 105 due to the fact that the latter are colored with a second color that is different from the first color. Other examples of color-coding will be obvious to those of ordinary skill in the art having the benefit of this disclosure.

Where such color-coding is employed, upon removing the gown (100) from a laundry machine a user will intuitively understand how to assemble the gown by connecting closing elements attached to the left border 901 and the right border 902 of the first sleeve 104 together due to the fact that the left border 901 and the right border 902 of the first sleeve 104 are of a common color. Similarly, the user will intuitively connect closing elements attached to the left border 1001 and the right border 1002 of the second sleeve 105 together due to the fact that the latter are colored with a second color that is different from the first color.

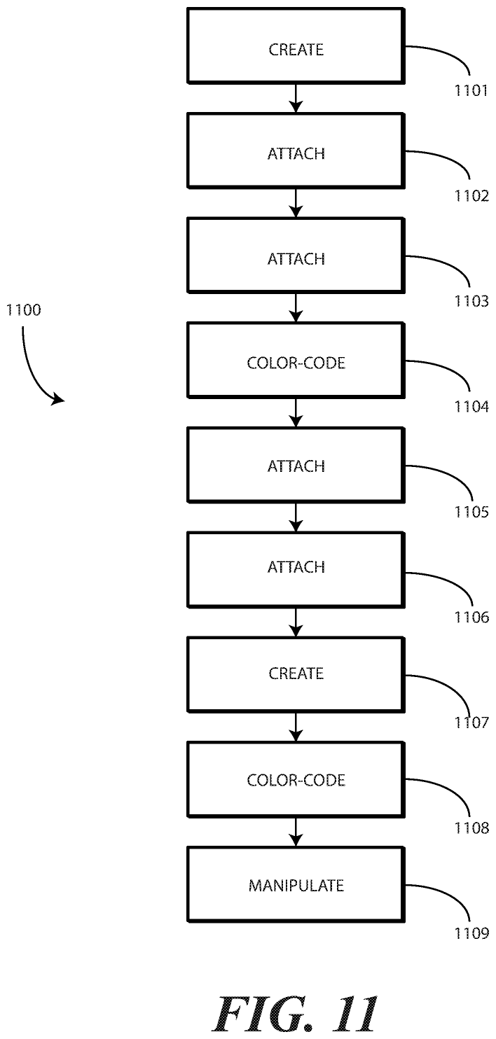

Turning now to FIG. 11, illustrated therein is one explanatory method 1100 for manufacturing a gown configured in accordance with one or more embodiments of the disclosure. Beginning at step 1101, an aperture is created in a front portion of a body-covering portion of a gown. In one embodiment, the body-covering portion includes a rear portion in addition to the front portion. In one or more embodiments, the rear portion defines an opening that extends from the base of the gown up the rear portion to a head insertion aperture.

In one or more embodiments, the body-covering portion includes an exterior side that is exposed outwardly and an interior side that is exposed inwardly. In one or more embodiments, the body-covering portion is configured to wrap about the torso of a wearer.

In one or more embodiments, the aperture created at step 1101 allows medical personnel to access the chest of a patient wearing the gown. In one or more embodiments, the aperture is generally rectangular, and has a width of between six and ten inches, such as about eight inches in one embodiment, and a height of between twelve and twenty inches, such as about fifteen inches in one embodiment. In one or more embodiments, step 1101 comprises creating a hem about the perimeter of the aperture to prevent the body-covering portion from fraying or tearing.

Step 1102 comprises attaching one or more fasteners to the body-covering portion such that the opening and head insertion aperture can be closed. In one embodiment, the fasteners comprise ties and/or a neck tie. In one embodiment, step 1102 comprises coupling a first tie to a first closure side edge of the opening, and coupling a second tie to a first side portion of the body-covering portion. Step 1102 can include coupling an extra tie on the interior side of the body-covering portion to allow the opening to be closed when the body-covering portion is turned inside out.

At optional step 1103, sleeves are attached to a body-covering portion. In one or more embodiments, the sleeves are short sleeves. However, the sleeves attached at step 1103 could be long sleeves as well. In other embodiments, step 1103 will be omitted and the resulting gown will have no sleeves at all.

In one or more embodiments, the sleeves attached at step 1103 are configured so that they can be opened to allow medical personnel to access a patient's arm when wearing the gown. These openings can extend the length of teach sleeve and may include closing mechanisms, such as snaps, that can selectively be coupled together to close the opening or uncoupled to open the opening.

Optional step 1104 comprises color-coding either the sleeves or a border to which the closing mechanisms are attached. In one or more embodiments, step 1104 comprises coloring the left border and the right border of the first sleeve attached at step 1103 with a first color, and coloring the left border and the right border of the second sleeve attached at step 1103 with a second color that is different from the first color. Where step 1104 is included, this color-coding advantageously functions to identify which closing element disposed along which border is normally coupled to a complementary closing element.

Step 1105 comprises attaching a flap to the exterior side of the body-covering portion. In one or more embodiments, step 1105 comprises attaching a base of the flap to the exterior side of the body-covering portion with a seam.

In one or more embodiments, the flap attached at step 1105 comprises one or more fasteners, such as snaps, which selectively attach the top portion of the flap to the neckline of the head insertion aperture where the sleeves intersect the head insertion aperture. In other embodiments the snaps can be replaced by other fasteners, including hook and loop fasteners, zippers, and so forth.

Step 1106 comprises attaching side ties to the flap and the body-covering portion that allow the flap to be coupled to the body-covering portion at the armpit-covering region where the sleeves and body-covering portion intersect. Step 1106 can comprise attaching a first tie to the body-covering portion and a second tie member to the flap on both the left and right sides of the flap.

In one or more embodiments, step 1106 comprises attaching the flap only to the exterior side of the body-covering portion. In one or more embodiments, no flap is attached to the interior side of the body-covering portion.

Step 1107 comprises creating a double-paneled telemetry pocket along major faces of the flap. In one or more embodiments, step 1107 comprises creating an aperture in the flap through which wires or other connector or tubes may be threaded through the flap. In one or more embodiments, step 1107 comprises creating a horizontal slit with abutting sides in the flap. However, in other embodiments step 1107 comprises creating a vertical slit with abutting sides. In still other embodiments, step 1107 comprises creating an open aperture with sides that do not abut. In still other embodiments, step 1107 comprises creating overlapping flaps that close the aperture, but that can be separated to open the same.

Step 1107 then comprises attaching first pocket panel on a first side of the flap such that it covers the aperture. Step 1107 then comprises attaching a second pocket panel on a second side of the flap such that it covers the aperture as well. In one or more embodiments, the first pocket panel and the second pocket panel attached at step 1107 are coupled at a three-sided connection location on a first side of the flap and on the second side of the flap.

In one or more embodiments, the flap attached at step 1106 is configured to substantially cover the chest portion of the body-covering portion, and is to cover the aperture created at step 1101 such that the second pocket portion attached to the flap at step 1107 is visible through the aperture created at step 1101 when the body-covering portion is turned inside out.

Optional step 1108 can comprise color-coding and/or patterning the exterior side of the body-covering portion differently from the interior side of the body-covering portion to indicate to which side the flap is attached. For example, the interior surface of the body-covering portion can be a first color while the exterior surface of the body-covering portion is a second color. In another embodiment, the interior surface of the body-covering portion can be configured in a first pattern, while the exterior surface of the body-covering portion can be a second pattern. Of course, combinations of patterns and colors can be used to identify to which side the flap is attached. Moreover, the flap attached at step 1106 can be color-coded and/or patterned at step 1108 so as to be a different color or pattern (or combination of both) to indicate to which side the flap is attached.

In still other embodiments, step 1108 can include applying printing to the exterior side of the body-covering portion and/or on the outside of the flap to indicate to which side the flap is attached. Similarly, in one or more embodiments step 1108 can comprise applying printing to the interior side of the body-covering portion and/or on the inside of the flap to indicate to which side the flap is attached.

At step 1109, the gown can be manipulated as described above with reference to FIGS. 1-7. For example, the gown can be manipulated by fastening the one or more fasteners and turning the body-covering portion inside out such that an interior side of the body-covering portion is exposed outwardly, thereby revealing a pocket panel of the double-paneled telemetry pocket through an aperture defined by the body-covering portion.

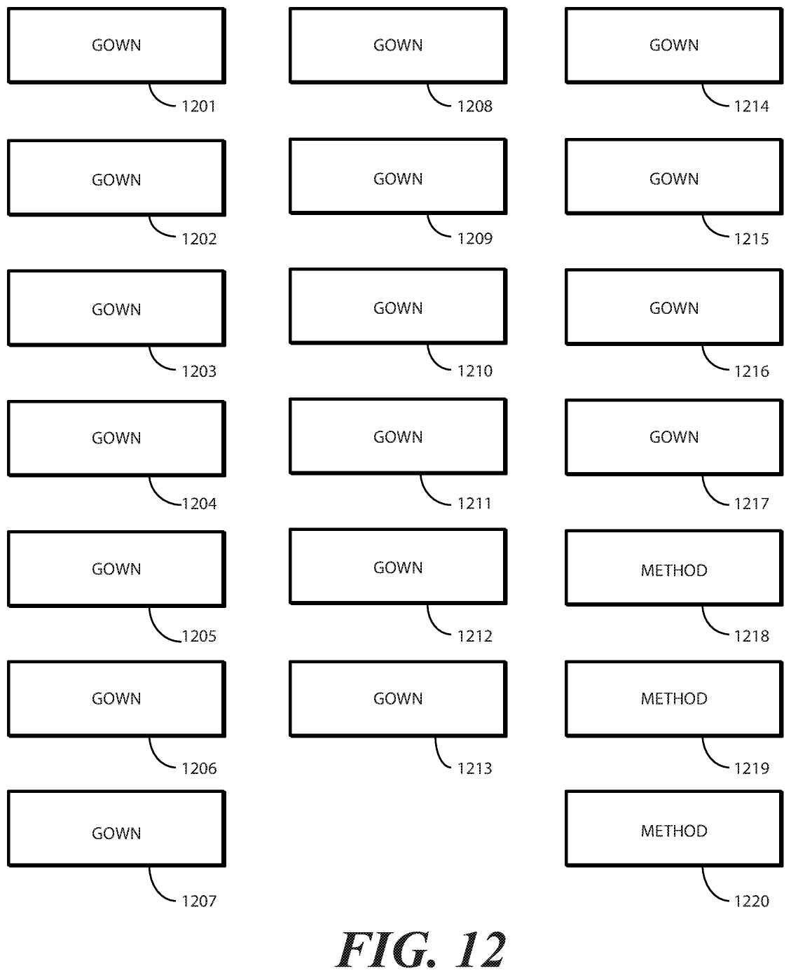

Turning now to FIG. 12, illustrated therein are various embodiments of the disclosure. At 1201, a gown comprises a body-covering portion having an interior side and an exterior side. At 1201, the body-covering portion defines an aperture.

At 1201, a flap is coupled to the exterior side. At 1201, the flap comprises one or more sides that are selectively attachable to the exterior side by one or more fasteners. At 1201, the flap further comprises a double-paneled telemetry pocket.

At 1201, the flap covers the aperture when the one or more fasteners are fastened. At 1201, the flap exposes the aperture when the one or more fasteners are unfastened and the flap is folded away from the exterior side.

At 1202, the exterior side of 1201 designates a high-risk side. At 1203, the interior side of 1202 designates a low risk side. At 1204, the gown of 1203 further comprises visual indicia indicating the exterior side is the high-risk side. At 1205, the gown of 1203 is color-coded to indicate the exterior side is the high-risk side.

At 1206, a first pocket panel of the double-paneled telemetry pocket of 1201 is visible through the aperture when the one or more fasteners are fastened and the body-covering portion is folded such that the interior side is exposed outwardly. At 1207, a second pocket panel of the double-paneled telemetry pocket of 1206 is visible when the one or more fasteners are fastened and the body-covering portion is folded such that the exterior side is exposed outwardly. At 1208, the first pocket panel of 1207 is visible when the one or more fasteners are unfastened, the flap is folded away from the exterior side, and the body-covering portion is folded such that the exterior side is exposed outwardly.

At 1209, the one or more fasteners of 1208 comprise at least two snaps and at least two ties. At 1210, the at least two ties of 1209 are situated between a base of the flap secured to the body-covering portion and the at least two snaps. At 1211, the at least two snaps of 1210 selectively attach a top portion of the flap to a neckline of the gown.

At 1212, the at least two ties of 1211 couple a left side of the flap and a right side of the flap to armpit-covering regions of the gown. At 1213, a portion of the flap of 1212 is visible through the aperture when the one or more fasteners are fastened and the body-covering portion is folded such that the interior side is exposed outwardly.

At 1214, a gown comprises a body-covering portion having an interior side and an exterior side. At 1214, the body-covering portion defines a chest access aperture.

At 1214, a flap is coupled to the exterior side. At 1214, the flap comprises one or more sides that are selectively attachable to the exterior side by one or more fasteners. At 1214, unfastening the one or more fasteners and folding the flap away from the exterior side reveals the chest access aperture.

At 1215, the flap of 1214 comprises a double-paneled telemetry pocket. At 1215, the double-paneled telemetry pocket comprises a first pocket panel coupled to the first side of the flap. At 1215, the first pocket panel covers a telemetry aperture defined by the flap.

At 1215, the double-paneled telemetry pocket also comprises a second pocket panel coupled to a second side of the flap. At 1215, the second pocket panel also covers the telemetry aperture.

At 1216, the second pocket panel of 1215 is visible through the chest access aperture when the one or more fasteners are fastened and the body-covering portion is folded such that the interior side is exposed outwardly. At 1217, the second pocket panel of 1216 is visible when the one or more fasteners are unfastened, the flap is folded away from the exterior side, and the body-covering portion is folded such that the exterior side is exposed outwardly.

At 1218, a method of constructing a gown comprises creating a double-paneled telemetry pocket along major faces of a flap. At 1218, the method comprises attaching a base of the flap to an exterior side of a body-covering portion of the gown. At 1218, the flap comprises one or more fasteners that selectively attach a top portion of the flap to the exterior side of the body-covering portion. At 1218, the method comprises fastening the one or more fasteners and turning the body-covering portion inside out such that an interior side of the body-covering portion is exposed outwardly, thereby revealing a pocket panel of the double-paneled telemetry pocket through an aperture defined by the body-covering portion.

At 1219, the method of 1218 comprises color-coding the body-covering portion to indicate to which side of the body-covering portion the flap is attached. At 1220, the method of 1218 further comprises attaching one or more intravenous (IV) sleeves to the body-covering portion.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims.

For example, the gowns described above have inherent ornamental appearance characteristics that are distinct from their functionality. An ordinary observer seeing another gown that included the inherent ornamental appearance characteristics would think that the other gown is substantially the same as the gowns illustrated and described above. This is true even when the gowns are compared in the context of the prior art.

Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A gown, comprising:
a body-covering portion having an interior side and an exterior side and defining an aperture;
a flap coupled to the exterior side, the flap comprising one or more sides that are selectively attachable to the exterior side by one or more fasteners, the flap further comprising a double-paneled telemetry pocket;
wherein the flap covers the aperture when the one or more fasteners are fastened, and exposes the aperture when the one or more fasteners are unfastened and the flap is folded away from the exterior side.

2. The gown of claim 1, wherein the exterior side designates a high-risk side.

3. The gown of claim 2 wherein the interior side designates a low risk side.

4. The gown of claim 3, further comprising visual indicia indicating the exterior side is the high-risk side.

5. The gown of claim 3, wherein the gown is color-coded to indicate the exterior side is the high-risk side.

6. The gown of claim 1, wherein a first pocket panel of the double-paneled telemetry pocket is visible through the aperture when the one or more fasteners are fastened and the body-covering portion is folded such that the interior side is exposed outwardly.

7. The gown of claim 6, wherein a second pocket panel of the double-paneled telemetry pocket is visible when the one or more fasteners are fastened and the body-covering portion is folded such that the exterior side is exposed outwardly.

8. The gown of claim 7, wherein the first pocket panel is visible when the one or more fasteners are unfastened, the flap is folded away from the exterior side, and the body-covering portion is folded such that the exterior side is exposed outwardly.

9. The gown of claim 8, the one or more fasteners comprising at least two snaps and at least two ties.

10. The gown of claim 9, the at least two ties situated between a base of the flap secured to the body-covering portion and the at least two snaps.

11. The gown of claim 10, the at least two snaps selectively attaching a top portion of the flap to a neckline of the gown.

12. The gown of claim 11, the at least two ties coupling a left side of the flap and a right side of the flap to armpit-covering regions of the gown.

13. The gown of claim 12, wherein a portion of the flap if visible through the aperture when the one or more fasteners are fastened and the body-covering portion is folded such that the interior side is exposed outwardly.

14. A gown, comprising:
a body-covering portion having an interior side and an exterior side and defining a chest access aperture;
a flap coupled to the exterior side, the flap comprising one or more sides that are selectively attachable to the exterior side by one or more fasteners;
wherein unfastening the one or more fasteners and folding the flap away from the exterior side reveals the chest access aperture.

15. The gown of claim 14, the flap comprising a double-paneled telemetry pocket comprising:
a first pocket panel coupled to a first side of the flap and covering a telemetry aperture defined by the flap; and
a second pocket panel coupled to a second side of the flap and covering the telemetry aperture.

16. The gown of claim 15, the second pocket panel being visible through the chest access aperture when the one or more fasteners are fastened and the body-covering portion is folded such that the interior side is exposed outwardly.

17. The gown of claim 16, the second pocket panel being visible when the one or more fasteners are unfastened, the flap is folded away from the exterior side, and the body-covering portion is folded such that the exterior side is exposed outwardly.

18. A method of constructing a gown, the method comprising:
creating a double-paneled telemetry pocket along major faces of a flap;
attaching a base of the flap to an exterior side of a body-covering portion of the gown, the flap comprising one or more fasteners that selectively attach a top portion of the flap to the exterior side of the body-covering portion; and
fastening the one or more fasteners and turning the body-covering portion inside out such that an interior side of the body-covering portion is exposed outwardly, thereby revealing a pocket panel of the double-paneled telemetry pocket through an aperture defined by the body-covering portion.

19. The method of claim 18, further comprising color-coding the body-covering portion to indicate to which side of the body-covering portion the flap is attached.

20. The method of claim 18, further comprising attaching one or more intravenous (IV) sleeves to the body-covering portion.
